(19) United States
(12) Patent Application Publication
(10) Pub. No.: US 2010/0167406 A1
(43) Pub. Date: Jul. 1, 2010

(54) MEASURING APPARATUS AND METHOD FOR DETERMINING FLUID PARAMETERS PROVIDED BY A LABORATORY SYSTEM

(76) Inventors: WOLFGANG STREIT, Hallein (AT); Gerald Probst, Wals (AT); Juha Koota, Salzburg (AT); Gyoergy Wenczel, Seekirchen (AT)

Correspondence Address: NOTARO, MICHALOS & ZACCARIA P.C., 100 DUTCH HILL ROAD, ORANGEBURG, NY, 10962, US

(21) Appl. No.: 12/617,237

(22) Filed: Nov. 12, 2009

(30) Foreign Application Priority Data
Nov. 13, 2008 (CH) 01772/08

(51) Int. Cl. G01N 31/00; G01F 1/00; G01L 7/00; G01N 33/00; G01N 7/00; G01N 1/00

(52) U.S. Cl. 436/2; 702/45; 702/50; 422/68.1; 422/82.13; 436/174

(57) ABSTRACT

Relates to a measuring apparatus (4) and corresponding methods carried out in e.g. hybridization systems for determining fluid parameters provided by a laboratory system (1). The measuring apparatus (4) comprises a measuring unit (5) and a processing unit (6) for determining the fluid parameters provided by the laboratory system (1) and the measuring unit (5) is accomplished to be able to be integrated into this laboratory system (1). The measuring apparatus (4) according to the invention is characterised in that the measuring unit (5) comprises a measuring block (15) without a reaction room (34) and at least one sensor (17). The measuring block (15) comprises hollow spaces (16) for receiving or guiding fluids (13) provided by the laboratory system (1). The at least one sensor (17) is arranged, for determining physical and/or chemical parameters of fluids (13) located in the hollow spaces (16), on or in fluidic operative connection with these hollow spaces (16) of the measuring block (15).

### RELATED PATENT APPLICATION

This patent application claims priority of the Swiss Patent Application No. CH 01772/08 filed on Nov. 13, 2008, the disclosure of which is herein incorporated by reference in its entirety and for all purposes.

### FIELD OF TECHNOLOGY

The invention relates, according to the preamble of independent claim 1, to a measuring apparatus with a measuring unit and a processing unit for determining fluid parameters provided by a laboratory system. This measuring unit is accomplished to be integratable into this laboratory system. The invention additionally relates, according to the preamble of independent claim 20, to a method for determining fluid parameters provided by a laboratory system. The measuring apparatus cited at the outset is used for carrying out this method.

The automation of laboratory processes in life science sectors, such as for example in pharmacological research, clinical diagnostics or genomics, is an important step in order to increase efficiency, quality and reliability of biochemical reactions and tests. Automated laboratory systems are known for carrying out a broad range of processes, such as for example for handling relatively large volumes of liquids (such as fermenters or pipetting apparatuses) or relatively small volumes of liquids (such as the spotting/immobilising of biological samples on supports), for nucleic acid amplifications (such as polymerase chain reaction/PCR, or sequencing reactions) or else for carrying out hybridisation reactions.

The problems which can arise in automated laboratory processes will be explained hereinafter by way of example based on the carrying-out of hybridisation reactions. Hybridisation reactions of this type are preferably carried out in gap-shaped, small spaces. They are binding reactions between two different chemical or biological binding partners. In this case, one of the partners, the sample to be hybridised, is typically immobilised on a solid substrate surface. These samples to be hybridised are then contacted with a suspension containing the desired binding partner, the specimen. Hybridisation reactions form the basis for a broad range of testing techniques in molecular biological laboratories. Immobilised samples can for example comprise amino acid-containing (e.g. proteins, peptides) or nucleic acid-containing (e.g. DNA, cDNA, RNA) samples. Specimens added to the immobilised samples may be any desired molecules or chemical compounds (e.g. DNA, cDNA and/or proteins or polypeptides) which hybridise with the immobilised samples or are otherwise connected thereto. Apparatuses or systems for carrying out hybridisation reactions of this type in an automated manner are already available.

The DNA microarray technique has, in particular, becoming established for hybridising DNA. This technique is based on a hybridisation reaction in which thousands of genes are detected and/or analysed at the same time or simultaneously. This technique includes the immobilisation of DNA samples from many genes on a substrate, e.g. on a glass object carrier for a light microscope. The DNA samples are preferably applied to the substrate in a defined array of sample spots, i.e. in a two-dimensional grid arrangement. Later, it is possible to conclude—starting from a specific position within an array of this type—the origin of the corresponding DNA sample and thus its identity. The technique further includes contacting the DNA sample array with RNA specimen suspensions or solutions in order thus to detect specific nucleotide sequences in the DNA samples. RNA specimens may be provided with what is known as a tag or label, i.e. a molecule which emits e.g. a fluorescence light having a specific wavelength.

Under good experimental conditions, RNA specimens hybridise or bind for example to immobilised DNA samples and form together therewith hybrid DNA/RNA strands. The more closely an RNA specimen matches a spotted DNA sample—i.e. the more perfectly the corresponding base pairs are complementary to one another—the stronger is the bond between them. The differences in the binding/hybridisation of RNA specimens to the various DNA samples of an array can be determined by measuring the intensity and the wavelength dependency of the fluorescence of each individual microarray element. Thus, it is then possible to find out whether and to what extent the degree of gene expression in the DNA samples tested varies. The use of DNA microarrays thus allows comprehensive statements to be made concerning the expression of large quantities of genes and concerning the expression pattern thereof, despite the fact that only small amounts of biological material have to be used.

### RELATED PRIOR ART

DNA microarrays have become established as successful tools. The laboratory systems for carrying out hybridisations have been improved on an ongoing basis (cf. e.g. U.S. Pat. No. 6,238,910 or document EP 1 260 265 B1 in the name of the Applicant of the current patent application). These documents disclose systems with devices for providing a hybridising space for the hybridisation of nucleic acid samples, proteins or tissues on an object carrier. A known standard device of this type forms with the object carrier a gap-shaped hybridisation space. The known standard device is depicted in FIG. 1 and will be described in greater detail hereinafter.

It can occur that a laboratory process which is carried out in an automated manner using a laboratory system, such as for example a hybridisation reaction of this type, yields results which are unclear or quite impossible to analyse. However, in such cases, it is not possible, or it is possible only to a limited extent, to ascertain which problems have led to such a poor result, and where the problems were caused (for example on the part of the user or in the laboratory system). In order nevertheless to achieve an acceptable result, it must be possible to recognise and eliminate the problem. The quality, efficiency and reliability of laboratory processes or biochemical reactions are in this case dependent on a broad range of parameters influencing the experimental conditions. Examples of parameters of this type are, in a hybridisation reaction, chemical parameters of the reagents which are specially adapted for the reaction (e.g. salt content of the washing buffers, as well as the pH, to which a hybridising medium is exposed/“application parameters”) and physical parameters such as the temperature or the pressure of the fluids located in the hybridisation system. In order to recognise an existing problem in a reaction, individual parameters would have to be altered in a targeted manner. However, the large number of parameters gives rise to very many different operating conditions which would all have to be tested out in a complex manner. Such detailed problem recognition and problem solving therefore require a lot of time to be invested. In addition, mishandling of the laboratory system can cause additional sources of error; this greatly increases the effort still further.

Biological/biochemical and physical approaches have been proposed in order to be able to ascertain a problem in a targeted manner, for example in hybridisation reactions. Thus, for example, application document DE 100 18 036 A1 discloses a method using, for increasing the efficiency of a hybridisation reaction, a biological control system for the parameters which were applied. Temperature and time are, in particular, disclosed here as parameters to be varied.

It is also known in the art that externally applied parameters, such as for example temperature and pressure, can be monitored. Generally speaking, these parameters are monitored by means of sensors which are typically attached in or directly to the devices generating these parameters such as heating apparatuses or pumps. Thus, U.S. Pat. No. 6,238,910 proposed that, in an automated hybridisation device, temperature sensors be directly integrated into a temperature plate. The temperature of this plate is transmitted to an object carrier by means of various intermediate layers (pads). Thus, the temperature is measured directly in the device that generates it.

An automated hybridisation device is known from WO 03/106033, in which an externally applied temperature is measured in or on a hybridisation space of an experimental unit. For this purpose, a thermoelement can be integrated into a cover part of this experimental unit.

### OBJECT AND SUMMARY OF THE PRESENT INVENTION

The object of the present invention is to propose alternative possibilities for ascertaining and analysing problems which can occur in automated laboratory processes and produce poor process results.

This object is achieved, according to a first aspect and the features of independent claim 1, by a measuring apparatus with a measuring unit and a processing unit, the measuring apparatus, for determining fluid parameters provided by a laboratory system, being accomplished to be integratable into this laboratory system. The measuring apparatus according to the invention is characterised in that
(a) the measuring unit comprises a measuring block without a reaction space and at least one sensor;
(b) the measuring block comprises hollow spaces for receiving fluids provided by the laboratory system, the hollow spaces being arranged substantially completely within the measuring block; and in that
(c) the at least one sensor is arranged, for determining physical and/or chemical parameters of fluids located in the hollow spaces, on or in fluidic operative connection with these hollow spaces of the measuring block.

This object is achieved, according to a second aspect and the features of independent claim 20, in that a method is provided that uses, for determining fluid parameters provided by a laboratory system, a corresponding measuring apparatus with a measuring unit and a processing unit. The measuring unit of this measuring apparatus is in this case integrated into the laboratory system. This method according to the invention is characterised in that
(a) the measuring unit comprises a measuring block without a reaction space and at least one sensor;
(b) the measuring block comprises hollow spaces which receive fluids provided by the laboratory system and which are arranged substantially completely within the measuring block; and
(c) physical and/or chemical parameters of the fluids located in the hollow spaces are determined by the at least one sensor which is arranged on or in fluidic operative connection with these hollow spaces of the measuring block.

The present invention thus provides a method and a measuring apparatus for carrying out the method which can be used to determine, in particular, fluid parameters provided by a laboratory system. This allows an effective analysis of results by means of processing of the signals which are delivered by the corresponding sensors and represent indirect information concerning apparatus parameters of laboratory systems. In the context of the present invention, gases, liquids and gas/liquid mixtures shall count as fluids.

Additional inventive features and preferred embodiments emerge from the dependent claims.

Advantages of the present invention include:
Laboratory apparatuses which are capable of carrying out laboratory processes of this type in an automated manner can be set, after production thereof, to preferred factory parameters by means of the proposed apparatus and method.
An apparatus and a method are proposed allowing problems to be ascertained in laboratory processes which are carried out in an automated manner.
Detected problems can be analysed in relation to their origin, for example in the laboratory apparatus itself or in the application.
Error detection and error analysis by means of the proposed apparatus or method allows prompt and thus time-saving and cost-reduced error allocation.

### BRIEF INTRODUCTION OF THE DRAWINGS

The invention will now be described in greater detail with reference to exemplary embodiments and schematic drawings. In this case, the drawings and description are not intended to limit the scope of the invention. In the drawings:

FIG. 1 is a perpendicular longitudinal section through a hybridisation unit known in the art of a hybridisation system with a standard device, instead of which a measuring unit according to the invention can be inserted into the hybridisation unit;

FIG. 2 is a perpendicular longitudinal section through a measuring unit which is inserted into the hybridisation unit of a hybridisation system and thus integrated into this hybridisation system;

FIG. 3 shows an exemplary measurement of a flow of fluid in a measuring section between two flow sensors 19 of a measuring unit according to the invention;

FIG. 4 shows highly simplified measuring designs of a measuring apparatus with a measuring unit and a processing unit, wherein:

FIG. 4A shows a first variant of the measuring design,

FIG. 4B shows a second variant of the measuring design, and

FIG. 4C shows a third variant of the measuring design;

FIG. 5 shows exemplary groups made up of hybridisation units in which a measuring unit is integrated instead of a standard device, wherein:

FIG. 5A shows a first variant of a group made up of four hybridisation units, in which a measuring unit is integrated instead of a first standard device,

FIG. 5B shows a second variant of a group made up of four hybridisation units, in which a measuring unit is integrated instead of a second standard device,

FIG. 5C shows a third variant of a group made up of two hybridisation units, in which a measuring unit is integrated instead of a third standard device, and

FIG. 5D shows a fourth variant of a group made up of two hybridisation units, in which a measuring unit is integrated instead of a third standard device.

### DETAILED DESCRIPTION OF THE INVENTION

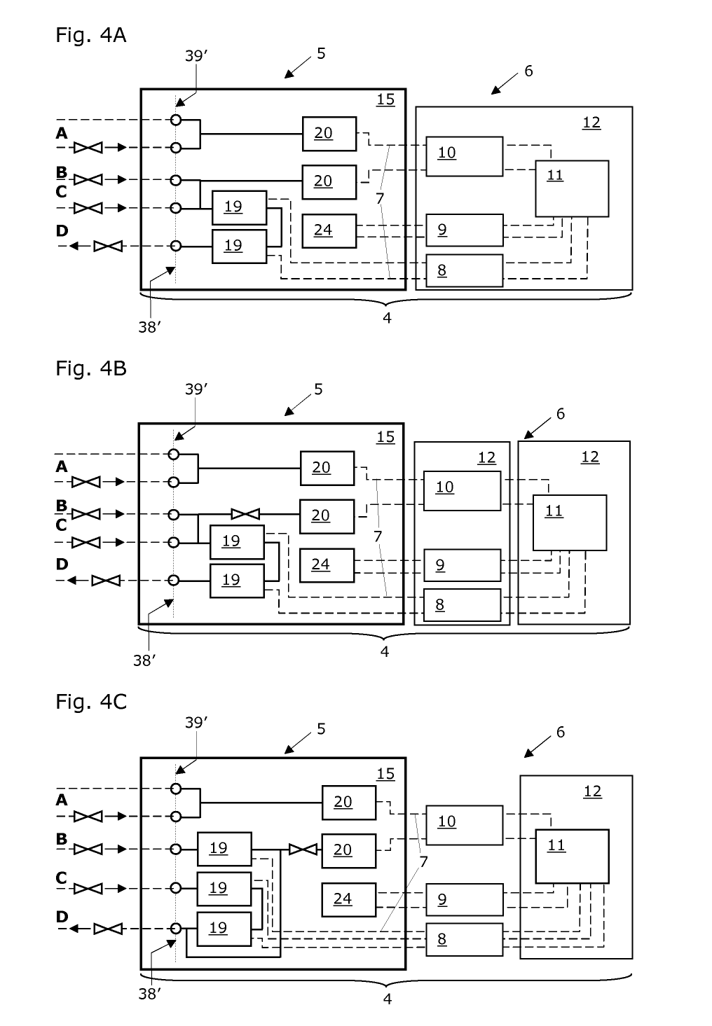

A measuring apparatus 4 according to the invention comprises a measuring unit 5 and a processing unit 6 (cf. FIG. 4). The measuring unit 5 is accomplished in such a way that it can be integrated into a laboratory system 1 in order to determine there the fluid parameters provided by the laboratory system 1. The term “laboratory systems 1” is in this case intended to refer to systems of the type allowing a broad range of laboratory processes to take place. In this case, it is preferable for the laboratory processes to be carried out in an automatable manner by means of these laboratory systems 1. Exemplary laboratory systems 1 can be accomplished to handle relatively large or relatively small volumes of liquids. These are known as fermenters or pipetting apparatus (relatively large volumes), or else as systems for the spotting/immobilising of e.g. biological samples on laboratory-typical supports. Other conceivable laboratory systems 1 are systems for carrying out PCR or sequencing reactions or, in a particularly preferred embodiment, systems for carrying out hybridisation reactions. The present invention will be described in greater detail based on the example of hybridisation systems 2, without thereby limiting the scope of the invention.

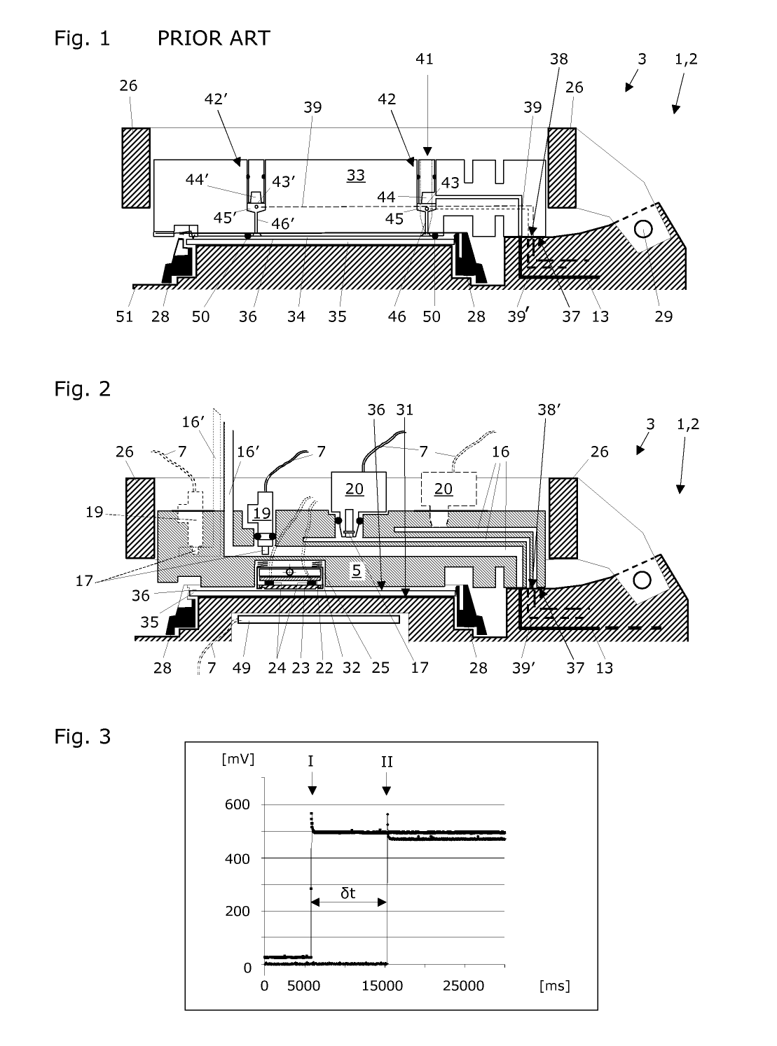

FIG. 1 is a perpendicular longitudinal section through a hybridisation unit 3, which is already known in the art, of a hybridisation system 2 of this type. The hybridisation unit 3 comprises a standard device 33 and is known from document EP 1 260 265 B1 or else from document EP 1 614 466 A2. Both documents are patent specifications or patent applications in the name of the Applicant of the current application and are herein incorporated by reference in their entirety. This standard device 33 is accomplished as a cover which can be moved relative to an object carrier 35. Typically, such an object carrier 35 comprises nucleic acid samples, proteins or tissue sections which are to be brought into contact (hybridised) with a specimen. They are immobilised on a surface 36 of the object carrier 35. Typical object carriers 35 may be glass object carriers 35 which are suitable for light microscopy or have dimensions which at least approximate the dimensions of glass object carriers, even though they are made of a different material (e.g. plastics material). Also known are glass or plastics material-based object carriers to which, for example, a cellulose membrane is fastened. For movement, the standard device 33 can be inserted into a holder 26. This holder 26 is then moved—with the standard device 33 inserted—relative to the object carrier 35 via an axis 29.

The standard device 33 defines with the object carrier 35 a gap-shaped hybridisation room 34. The object carrier 35 can in this case be positioned on a frame 28. The frame 28 can serve both to position object carriers 35 within a hybridisation unit 3 and to transport or to store the object carriers 35. The frame is itself positioned on a base plate 51 of the hybridisation unit 3. For sealing the hybridisation room 34, the standard device 33 comprises a sealing face 50 which is preferably embodied as an annular seal, for example as an O-ring. The sealing face 50 seals the hybridisation room 34 from the environment in that the sealing face 50 is abutted to a surface 36 of the object carrier 35.

The standard device 33 additionally comprises lines 39 for the feeding and discharging of media into the hybridisation room 34 and out of the hybridisation room 34 respectively. The standard device 33 further comprises a specimen supply line 41 which is accomplished for the feeding of specimen liquids into the hybridisation room 34, as well as an agitation device 42 for moving liquids in the hybridisation room 34. Possible embodiments are described in depth in the above-mentioned documents EP 1 260 265 B1 and EP 1 614 466 A2, so that, for details, reference is expressly made to these specifications. This agitation device 42 comprises for moving liquids a pressure chamber 44 in which an agitation pressure is generated. The pressure chamber 44 is separated from an agitation chamber 45 via a membrane 43. The agitation chamber 45 is, in turn, connected to the hybridisation room 34 via an agitation line 46. After establishing a thermal equilibrium in the hybridisation room 34 and after closing the specimen supply line 41, a fluid is intermittently brought into the pressure chamber 44 or let out therefrom via a pressure line. Depending on whether there is an excess or reduced pressure, the membrane 43 bends, thereby reducing or increasing the size of the agitation chamber 45 accordingly, and moves the liquid in the hybridisation room 34 via the agitation line 46. A variant of a standard device can comprise a second agitation device 42′ with a pressure chamber 44′, a membrane 43′, an agitation chamber 45′ and an agitation line 46′, so that both means can generate an oscillating movement of liquids in the hybridisation space.

As described in document EP 1 614 466 A2, the standard device 33 can comprise, in addition to the agitation device 42,42′, a pressure means 47 which is completely separate therefrom and which is used to generate a room pressure in the hybridisation room 34. This room pressure is increased over the surrounding atmospheric pressure and is superimposed by the agitation pressure in the hybridisation space. The room pressure serves to prevent or to suppress the formation of air bubbles in the hybridisation room 34.

Substantially all the lines 39 of the standard device for the feeding or discharging of media open out in a common connection plane 38 of the standard device 33. This connection plane 38 preferably extends substantially parallel to the hybridisation room 34. A connection plate 37 of the hybridisation unit 3 allows the lines 39 of the standard device 33 to be tightly connected to the lines 39′ of the hybridisation system 1.

The measuring apparatus 4 according to the invention and the connection thereof to a laboratory system 1 will now be described and commented on in greater detail below based on the example of a prior-art hybridisation system 2 as introduced above:

FIG. 2 is a perpendicular longitudinal section through an exemplary measuring unit 5 of a measuring apparatus 4 according to the invention in a highly simplified and schematised illustration. A measuring apparatus 4 according to the invention serves in this case to determine fluid parameters provided by a laboratory system 1. The measuring apparatus 4 according to the invention is also suitable for carrying out a method according to the invention. For this purpose, the measuring unit 5 of the measuring apparatus 4 is integrated into a laboratory system 1. As mentioned hereinbefore, the measuring apparatus 4 will be commented on in greater detail in relation to hybridisation systems 2, without being restricted to use in systems of this type.

According to the invention, the measuring unit 5 shown in FIG. 2 comprises a measuring block 15 and at least one sensor 17. In this case, the measuring block 15 is embodied in such a way that it does not provide any space for carrying out a laboratory process or a reaction or cannot form part of such a space. In the context of this invention, a reaction room 34 of this type will refer to a space in which a biological or chemical process (reaction) can take place. Processes of this type have already been discussed in detail above; hybridisation reactions or else PCR reactions may be mentioned by way of example at this point.

The at least one sensor 17 is arranged on or in fluidic operative connection with hollow spaces 16 of the measuring block 15. These hollow spaces 16 serve to receive fluids 13 provided by the laboratory system 1 or the hybridisation system 2. This arrangement enables the sensors 17 to determine the physical and/or chemical parameters of the fluids located in the hollow spaces 16.

These hollow spaces 16 can be accomplished as fluid lines and/or as fluid chambers arranged substantially completely within the measuring block 15. Preferably, the hollow spaces 16 run out in a common connection plane 38′ of the measuring unit 5. The arrangement of the run-outs of fluid lines and fluid chambers in this connection plane 38′ corresponds substantially to an arrangement of feed and/or discharge lines in a common connection plane 38 of a laboratory system 1. By means of the connection plate 37 of the hybridisation system 2, in which the lines 39′ of the hybridisation system also run out into a plane, the hollow spaces 16 of the measuring unit 5 are tightly connected to the line system of the hybridisation system 2 and thus functionally integrated into the hybridisation system 2. In fact, the measuring units 5 according to the invention can easily be inserted into a hybridisation system 2 instead of a standard device 33.

For determining parameters of the fluids 13 located in the measuring block 15, the at least one sensor 17 is arranged on or in fluidic operative connection with the hollow spaces 16 of the measuring block 15. The at least one sensor 17 is preferably positioned either on or in the hollow spaces 16, so that it is in direct contact with the fluid 13 to be measured, without influencing the parameters of the fluids 13 themselves. Alternatively, the at least one sensor 17 is arranged in fluidic operative connection with the hollow spaces 16 of the measuring block 15. In this case, the sensor 17 is not necessarily in direct contact with the fluid 13 of the hollow spaces 16; but can be separated from the hollow spaces 16, for example by a membrane or another layer. The fluid parameters are then detected by the at least one sensor 17 via the fluidic operative connection. An arrangement of this type is indicated in FIG. 2 for a sensor 17 which is embodied as a pressure sensor 20 for gases. Fluid parameters, such as the flow or else fluid pressure, can then be detected by the sensor 17 via the fluidic operative connection.

The fluids 13 of the hybridisation system 2 that are received by the hollow spaces 16 may be liquids, gases or mixtures of liquids and gases. Liquids can include for example media, buffers or reagents for carrying out reactions. Liquids which are used can also include further reactants or reaction catalysts, such as for example enzymes or other proteins. Gases include air or else inert gases such as e.g. noble gases or nitrogen (N₂). In a preferred embodiment, N₂ is used as a drying fluid and pumped or blown through the hollow spaces 16. In particular in hybridisation reactions, N₂ is preferably used for drying hybridisation products on object carriers 35 or for blowing out the hollow spaces 16 and other lines 39,39′.

In accordance with this embodiment according to the invention of a measuring apparatus 4, the current parameters of fluids 13 provided by a laboratory system 1 are therefore determined, when the measuring apparatus is integrated into the laboratory system 1, under conditions which are at least approximately in step with actual practice within the measuring block 15: Not the parameters are measured that were generated on the fluid remote from the measuring block 15 by external apparatuses, such as e.g. pressure pumps or heating/cooling systems (required values). The measuring apparatus 4 therefore provides substantially comparable hollow spaces having similar volumes or flow resistances to those displayed by a standard device 33. As a result, those fluid parameters are determined that prevail within the laboratory system at the destination (actual values). The fluid parameters provided by external apparatuses provided may differ, after they have passed through all manner of lines and valves, to a greater or lesser extent from the parameters prevailing in the measuring block 15. However, the determination of the actual value is critical for the factually reliable estimation of a quality of a reaction. Based on this actual value, it is then possible, on the one hand, to draw well-founded conclusions concerning the reaction process which has taken place in the laboratory system. On the other hand, an experimenter, a service engineer or the manufacturer can for example use the actual value determination for calibrating and adjusting the external apparatuses.

Sensors 17 are well known in the art. In the context of this invention, in particular, the sensor 17 is understood to be technical components of the type that are capable of recording physical and/or chemical properties of the sensor's environment or of measured objects as measured values. This recording of measured values can be carried out qualitatively (for example as a yes/no answer) or quantitatively. The detected measured values can then be converted into processable variables, e.g. electric or electronic signals, by the sensor 17 itself or by means of further components which are connected downstream. Preferably, the measuring unit 5 comprises at least one sensor 17. However, depending on requirements, the measuring unit 5 can also comprise a plurality of sensors 17.

Examples of parameters to be detected include pressure, flowing rate, mass flow or volume flow, temperature, sonic conduction or density, optical properties (e.g. colouration or turbidity), or else substance concentrations or pHs. In principle, within the scope of this invention, it is possible to measure all physical and/or chemical properties of a fluid that are known to the person skilled in the art and can be determined by means of sensors 17. Preferably measured fluid parameters are the pressure, the volume flow (determined based on the flowing rate or the flow) and the temperature of a fluid.

The functioning of sensors 17 is sufficiently known in the art, and will therefore not be discussed any further at this point. Mention may be made here, by way of example, of the principle of semiconductor technology or else of resistance measuring. Sensors 17 of a broad range of modes of operation can be integrated in a measuring apparatus 4 according to the invention. The only prerequirement is the detection of the physical and/or chemical parameters of the fluids 13 located in the hollow spaces 16, and the conversion thereof into processable variables or signals. It is immaterial for the feasibility of the invention whether a type of sensor 17 is used only for one specific parameter or for a plurality of parameters. Thus, for example, a sensor can be used both for determining a flow rate of a liquid or else for determining a flow rate of a gas, if it is accomplished in an appropriate manner. Conventional commercial sensors 17 can generally be used.

The measurement signal emitted by the at least one sensor 17 is processed into a desired variable or response by a processing unit 6 of the measuring apparatus 4 for evaluation. The processing unit 6 comprises at least one microcontroller 11 which transmits the digital data received by the at least one sensor 17 to a computer 12 of the processing unit 6. Furthermore, the processing unit 6 comprises data routing systems 8,9,10 which prepare and forward the signals emitted by the sensor for the microcontroller 11. Data routing systems 8,9,10 of this type comprise analogue/digital converters 10, serial or parallel data buses 8 and direct digital input/output connections 9. It is also conceivable to use in the processing unit 6 other elements or methods which are known in the art and are necessary for controlled data forwarding and processing.

In a first preferred embodiment, the at least one sensor is accomplished for determining a flow rate of a fluid or for determining a fluid pressure. In this case, for determining a flow rate of a fluid, the at least one sensor 17 can be accomplished as a flow sensor 19 for liquids or gases. Furthermore, for determining a fluid pressure, the at least one sensor 17 can be accomplished as a pressure sensor 20 for liquids or gases.

Examples of sensors 17 used in this and the following embodiments include:

Pressure sensors:
From the company SensDev LTD. (47 Station Street, Birkirkara—BKR 12, Malta/Kressnerstr. 12, 09217 Burgstaedt, Germany):
SenSpecial™ pressure sensor SCPB-B0/3.5G50i2C32717R5 and/or
SenSpecial™ pressure sensor SCPB-B0/1.5G50i2C32717R5
   These exemplary pressure sensors are based on semiconductor technology and function in accordance with the piezoresistive principle.
   Fluid flow sensors:
   From the company IST AG (Industriestr. 2, 9630 Wattwil, Switzerland):
FS1.A.1L.195
   The measuring principle of this sensor is based on resistance measurement by means of a high-ohm resistor and a low-ohm resistor.
   Temperature sensors:
   From the company IST AG (Industriestr. 2, 9630 Wattwil, Switzerland):
TSic-306F
   This exemplary temperature sensor is based on measurement of a voltage which is linear to the temperature. This voltage is then digitised by an analogue/digital converter.

In a second preferred embodiment, the measuring unit 5 comprises at least two sensors 17. According to this second preferred embodiment, the measuring unit 5 is used to measure at least one pressure and a flow rate of fluids 13 provided by a laboratory system 1. For this purpose, one sensor is accomplished as the flow sensor 19 and the second sensor is accomplished as the pressure sensor 20. Depending on the tolerance of the sensors 19,20 which are used, the two regions of the hollow spaces 16 on which the sensors 19,20 are arranged are separated from each other by means of valves. This is desirable in particular when, for example, a gas pressure sensor 20 which is used is impaired in its functioning on contact with liquid. An embodiment of this type is illustrated by way of example in FIGS. 4B and 4C. These figures show various variants of measuring designs with a measuring unit 5 and a processing unit 6. A separation of hollow spaces 16 was for example achieved using the valve NEX 212S from the company Parker Hannifin Corporation (distributed by Sensortechnics GmbH, Boschstrasse 10, 82178 Puchheim, Germany). In the embodiments illustrated here, the pressure sensor SCPB-B0/3.5G50i2C32717R5 from the company SensDev LTD. was in this case used for determining the gas pressure of a drying fluid (e.g. N₂). This sensor 17 is accomplished as a high-pressure sensor 20 and is therefore particularly suitable for determining the N₂ pressure. In order to protect the sensor from liquid pressure surges, the use of a valve was introduced in these variants.

A particularly preferred variant is the use of separate pressure sensors 20 for different pressure ranges, as indicated above. This variant is preferred when the measuring unit 5 is integrated, for example, into an above-described hybridisation system 2 from the prior art. In this case, a high-pressure sensor and a low-pressure sensor are used for determining an N₂ pressure and for determining an agitation and/or chamber pressure. High-pressure and low-pressure sensors are in this case characterised by different measuring ranges. A sensor arrangement of this type with two pressure sensors 20 is illustrated in FIGS. 4A, 4B and 4C.

For determining the pressure of a drying fluid, such as N₂, a pressure sensor 20 which is accomplished as a high-pressure sensor (having a measuring range of up to 3.5 bar) is preferably used (for example the SenSpecial™ pressure sensor SCPB-B0/3.5G50i2C32717R5 from the company SensDev LTD.). The pressure of the drying fluid to be measured is in a preferred embodiment between 1.5 and 3.5 bar, in a particularly preferred embodiment between 2 and 3 bar and in a most particularly preferred embodiment between 2.5 and 2.9 bar above the surrounding normal pressure.

A pressure sensor 20 accomplished as a low-pressure sensor (having a measuring range of up to 1.5 bar) is, on the other hand, used for determining an agitation pressure and/or a room pressure. The room pressure is generated via the pressure means 47 and is preferably between 10 mbar and 1.5 bar above the surrounding normal atmospheric pressure. Particularly preferred is an agitation pressure of from 0.9 to 1 bar above the surrounding normal atmospheric pressure. The agitation pressure is formed, as described above, by means of the agitation device 42 independently of the room pressure. The agitation pressure is in this case superimposed on the room pressure in the hybridisation room 34. The agitation pressure is preferably between 0.5 and 1.4 bar, and particularly preferably between 1.2 and 1.3 bar. Thus, the pressure which is to be determined by the low-pressure sensor, which pressure is composed in particular of the chamber pressure and agitation pressure, is between 10 mbar and 1.5 bar above the surrounding normal pressure. A preferably used pressure sensor 20 embodied as a low-pressure sensor is the SenSpecial™ pressure sensor SCPB-B0/1.5G50i2C32717R5 from the company SensDev LTD.

This use of two separate sensors 20 for low pressure and high pressure of gases allows construction deficiencies in a hybridisation system 2 according to the invention to be ascertained, for example. If, for example, a membrane 43,43′ of the agitation device 42,42′ is defective, the drop in pressure can be specifically detected by the low-pressure sensor. Errors are therefore detected in a sensor-specific manner.

In an alternative embodiment, the measuring unit 5 comprises, for determining a flow rate of a fluid 13, at least two identically designed sensors 19,20 which are arranged set apart from one another on or in fluidic operative connection with a hollow space 16. Preferably, this hollow space 16 is embodied as a fluid line which corresponds in its dimensions to a fluid line of a laboratory system 1, e.g. a standard device 33 of a hybridisation system 2 described here. This alternative embodiment is preferred, in particular, for determining a liquid flow. The flow rate is in this case measured on a measuring section. This measuring section is that section of the fluid line that lies between the two flow sensors 19. The principle of a flow measurement of this type is that each of the two preferably identically constructed sensors 19 delivers a signal when a liquid front passes the sensor. For determining the flow rate, use is then made not of the amplitude of the signal, but of the time t (the time signal) that the liquid requires to flow through the measuring section between the two identically designed sensors 19. The volume flow is then calculated by factoring in the length of the measuring section and the diameter of the fluid line.

An exemplary measurement of the flow of fluid in the measuring section between the two flow sensors 19 was carried out by means of a prototype of the measuring block 15 of the measuring unit 5 according to the invention (cf. FIG. 2) and is illustrated in FIG. 3. In this case, the x-axis is the time axis which is divided into increments of 5,000 ms or 5 s. The y-axis shows the intensity or the amplitude of the flow sensor signal in [mV]. This illustration clearly shows that the time t that the liquid requires to flow through the measuring section between the two sensors 19 corresponds to the time difference (δt) illustrated in this figure, or is calculated therefrom.

The sensors FS1.A.1L.195 from the company IST AG were used for this signal measurement. These are identically designed flow sensors for gases or liquids. The signal that the sensor I and the sensor II deliver on passing of a liquid front may be seen very clearly. The clear signals allow the time (δt) that the liquid front requires to cover the measuring section between the two sensors I,II to be determined precisely and calculated back accordingly to the flow rate. Preferred for this hybridisation system are flow rates of liquids of between 5 ml/min and 20 ml/min, particularly preferably between 8 ml/min and 14 ml/min. Although this flow sensor 19, which is specifically suitable for a flow measurement of this type, can directly determine the flow rate of a liquid, experiments have revealed that the measurement is more accurate when two identically designed flow sensors 19 of this type are arranged on a measuring section and the flowing rate is concluded by means of a measured time signal. Possible disturbing influences of changing ambient conditions (e.g. changes in the room or fluid temperature) can be reduced in this way. However, in a variant of this embodiment, use may also be made of sensors 17 which are embodied as light barriers and which emit optical signals for further processing. A sensor of this type may be a conventional commercial forked light barrier.

An alternative embodiment of this type of a measuring unit 5 with two identically designed sensors for determining a flow rate of a liquid is illustrated in FIG. 2. Here, the measuring section is embodied in a tubular manner, and the two identically designed sensors 19 are arranged on or in fluidic operative connection with the tubular fluid line 16′, one at the beginning and one at the end of the tube. One side of the measuring section is illustrated by a solid line, the other side by a broken line. The start and end extend in this case substantially horizontally; the central part of the tube is illustrated as vertically extending fluid lines. Also conceivable, however, is any other arrangement outside and/or within the measuring unit 5, in which the two sensors 19 define a signal path. If the measuring unit 5 is integrated into an above-described hybridisation system 2, the dimensions of the measuring section within the measuring block are preferably selected in such a way that they correspond at least approximately to the dimensions of a line with the fluid to be characterised within the standard device 33.

As mentioned above, in a particularly preferred embodiment of this alternative arrangement, the sensors 17, which are identically designed for measuring a flowing rate of a fluid, are accomplished as flow sensors 19. The use of two light barriers 21 as two identically designed sensors 17 of this type is also preferred. A flow sensor 19 can in this case emit an acoustic or else electric-capacitive signal, while the sensors accomplished as light barriers 21 emit optical signals for further processing. The signals emitted by the sensors are then used by the processing unit 6 of the measuring apparatus 4 for calculating a flow rate of the fluid 13.

If the measuring apparatus 4 is integrated, in accordance with the invention, via its measuring unit 5 into a laboratory system 1, it can determine “on site” the fluid parameters provided by the laboratory system 1 (actual values). Preferably, the measuring unit 5 of the measuring apparatus 4 is inserted into a hybridisation system 2. The term “hybridisation system 2” is intended to refer, in the scope of the present invention, to a laboratory system 1 of the type that is suitable for carrying out hybridisation reactions. Typically, hybridisation systems 2 of this type provide at least one reaction room 34 in which the hybridisation reaction can take place. The hybridisation system furthermore comprises vessels for storing fluids 13, lines, pumps, valves, seals, apparatuses for generating fluid parameters and the like. An exemplary hybridisation system of this type is the above-mentioned system known in the art from documents EP 1 260 265 B1 or EP 1 614 466 A2. Preferably, the measuring unit 5 is integrated into a hybridisation unit 3 of this hybridisation system 2.

FIG. 2 is a simplified illustration of a hybridisation system 2 of this type, into which a measuring unit 5 of a measuring apparatus 4 according to the invention is integrated. As has been described and may be seen from FIG. 1, the hybridisation system 2 comprises a hybridisation unit 3 with a standard device, the standard device 33 defining with an object carrier 35 the hybridisation room 34. In FIG. 2, instead of the standard device 33, a measuring unit 5 of the measuring apparatus 4 according to the invention is inserted into the holder 26, so that the measuring unit 5 can be moved relative to the object carrier 35 or the bottom plate 51 by means of the holder 26. If the measuring unit 5 is to be used in a different laboratory system 1, it can be inserted into this system 1 in a different manner. Use may for example be made of simple plug-on or sliding mechanisms as well as of other mechanisms from the prior art which are well known to the person skilled and will therefore not be commented on any further here.

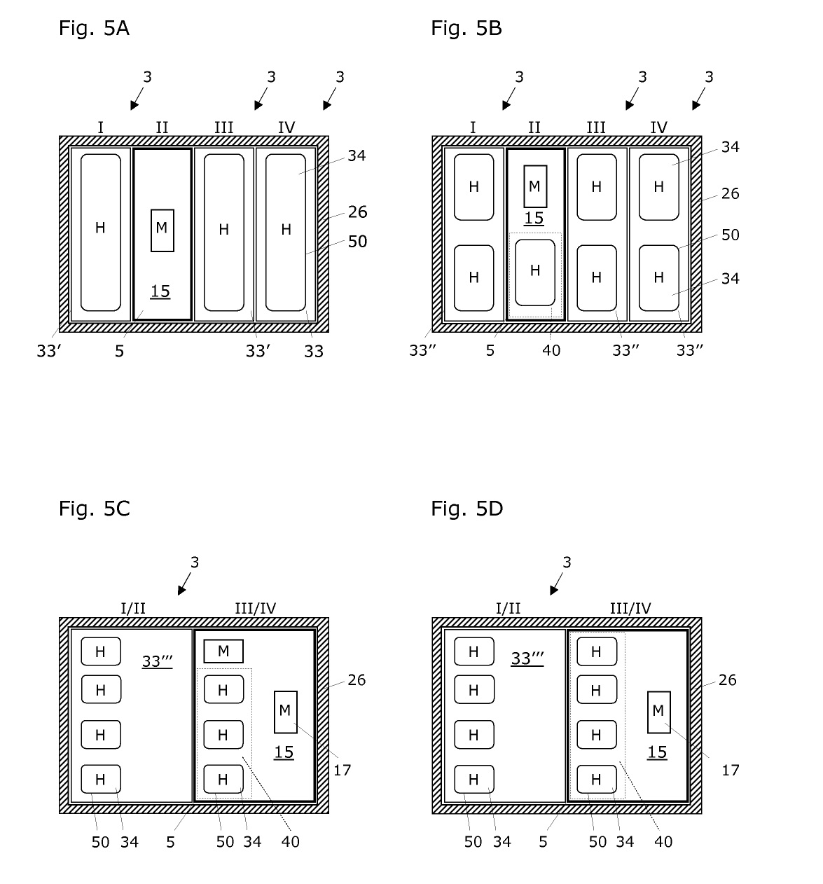

If the measuring unit 5 of the measuring apparatus 4 is inserted, as shown in FIG. 2, into a hybridisation system 2, no hybridisation reactions are carried out at this position, as the measuring unit 5 according to the previously described embodiments does not comprise or define a reaction room 34. If reactions are to be carried out in parallel with the measurement, preference is given to an arrangement of 2 or more hybridisation units 3, one of which is replaced by the measuring unit 5 (cf. FIG. 5A). In this way, reactions can be carried out by means of the hybridisation unit 3, and the parameters can be determined in parallel of the same fluids by means of the measuring apparatus 4. This is facilitated in that the feed lines and discharge lines 39′ of the hybridisation system 2 can be connected to the hollow spaces 16 of the measuring block 15 via connections of the measuring unit 5. In this case, substantially all the hollow spaces 16 of the measuring block 15 run out in a common connection plane 38′ of the measuring unit 5. Tight connecting of the hollow spaces 16 of the measuring unit 5 to the lines 39′ of the hybridisation system 2 is facilitated by means of the connection plate 37 of the hybridisation system 2.

A broad range of parameters are critical, in particular for hybridisation reactions. In addition to the flow rate and the pressure of fluids 13, which are necessary for an appropriate supply of fluid and thus for providing specific reactants, the temperature is also an important parameter. The temperature of a hybridisation reaction is determined by the temperature of the fluids 13 provided and also by the temperature of the object carrier 35. It is generally influenced via temperature regulators and heating elements 49. Temperature regulators of this type correspond, for example, to the temperature control plate of the hybridisation system 2 described here. The temperature of a temperature control plate of this type can in this case be adjusted via one or more heating elements 49. Peltier elements are preferred, although other heating elements well known to the person skilled in the art can also be used in this connection. In particularly preferred variants of the previously described embodiments, the measuring unit 5 of the measuring apparatus 4 therefore comprises a niche 25 in which at least one temperature sensor 24 is arranged. If a measuring unit 5 of this type is integrated into the hybridisation system 2, then the measuring unit is preferably embodied so as to be movable by means of the holder 26 toward a surface 31 of the hybridisation system 2, from which a temperature is to be determined. FIG. 2 shows a measuring unit 5 of this type in which, by way of example, two temperature sensors 24 are arranged in the niche 25. Examples of surfaces 31 of the hybridisation system include the surface 36 of an object carrier 35 or else the surface 52 of the base plate 51 of the hybridisation unit 3. Preferably, the temperature sensor 24 is or the two temperature sensors 24 are arranged on a printed circuit board 22 in the niche 25 in such a way that, during a movement of the measuring unit 5, the surface 31 is abutted by a metal plate 23. This metal plate 23 is preferably made from a metal having good thermal conductivity, such as e.g. aluminium or aluminium alloys, and is in good thermal contact with the temperature sensors 24 which are connected to the processing unit 6 via the printed circuit board 22. The metal plate 23 can have dimensions which essentially correspond to the face area of the niche 25, thus, not protruding over the lateral edges of the niche 25. In FIG. 2, the metal plate 23 shown is dimensioned so that it is partially arrangeable within the niche 25 (in view of its height). Alternatively, the metal plate 23 can be accomplished to be bigger than the area of the niche 25. The metal plate 23 would then protrude over the lateral edges of said niche 25. However, the metal plate 23 preferably is not bigger than the surface 31, on which it may act on.

In a particularly preferred variant, the at least one temperature sensor 24 of the measuring unit 5 is embodied so as to be able to be applied resiliently to a surface 31 of the hybridisation system 2, from which a temperature is to be determined. For this purpose, at least one spring element 32 is attached in the niche 25; however, depending on the form of the sensor 24 and niche 25, a plurality of spring elements 32 can also be used. This arrangement is advantageous, in particular when the temperature sensor is or the temperature sensors 24 are to be brought as close as possible to the surface 31 of the hybridisation system 2 without damaging this surface 31, e.g. by pressing-on too hard.

It is preferable for the surface 31 of the hybridisation system 2 to be accomplished as a temperature control plate. If the surface 31 is contacted directly by the temperature sensor 24, i.e. by the metal plate 23 thereof, then the temperature of the metal plate 23 adapts very quickly to that of the surface 31, so that the measurement can be carried out simply via touch contact and conduction of heat. Thus, the actually prevailing temperature of the surface 31 can be determined by means of the temperature sensor 24 of the measuring unit 5. If, on the other hand, the measurement is carried out by means of detection of heat radiation, the sensor 24 would not necessarily have to contact the surface 31, but could be arranged at a defined distance therefrom (not shown). Measurement of the temperature at the surface 31 based on convection would also be conceivable; nevertheless, this variant is inferior to the detection of heat radiation and measurement by means of touch contact and conduction of heat. Measurement by means of touch contact and conduction of heat is particularly preferred, because this measuring method can be carried out in a technically simple and inexpensive manner while still providing reliable test results.

In a particularly preferred variant, a temperature sensor 24 having a measuring range as broad as possible with an accuracy as high as possible is used for determining the temperature of a surface 31 of the hybridisation system 2. A temperature sensor 24 used in this particularly preferred variant is, for example, the temperature sensor TSic-306F from the company IST AG (Industriestr. 2, 9630 Wattwil, Switzerland). This sensor is used to generate a voltage which is linear to the temperature and is digitised by an analogue/digital converter. This sensor has a measuring range of from 0° C. to 100° C. with an accuracy of +/−0.1° C. to 0.3° C. However, two or more temperature sensors 24 can also be used for determining the temperature, each of these temperature sensors 24 used having a different measuring range with in each case high accuracy. Shortening the measuring section facilitates the necessary offset calibration.

Depending on the requirements and fluids used by the laboratory system 1, one or more sensors with specificity for a broad range of fluid parameters can be used for the measuring apparatus 4 according to the invention. Particularly preferred embodiments and variants of sensors and the arrangement thereof within the measuring apparatus have already been discussed in this specification and may also be seen from FIGS. 4A to 4C. These figures show various variants of measuring designs of a measuring apparatus 4 according to the invention. These measuring designs clarify the interconnectedness of the sensors and the computer for evaluating the sensor signals by means of various data routing systems 8,9,10 and at least one microcontroller 11. Typically, the microcontroller 11 is part of the computer 12. The sensor data are first transported to the data routing systems 8,9,10 via connections 7 and pre-processed, so that they can be evaluated by the microcontroller 11 and the computer 12. These data routing systems 8,9,10 are preferably comprised structurally by the computer 12, as illustrated in FIG. 4A. Alternatively, the data routing systems 8,9,10 are combined separately to form a structural unit which is independent of the computer 12 (FIG. 4C), or which is comprised by a second computer 12 (FIG. 4B).

FIG. 4 shows three preferred variants of the measuring design according to the invention. In each case, the connections or the feed and discharge lines 39′, which are provided by the hybridisation system 2, are shown respectively on the left side. This provision is carried out preferably in a common connection plane 38 (cf. FIG. 2) and is identical for all the variants in FIGS. 4A, 4B and 4C. A measuring apparatus 4 according to the invention comprises in each case a measuring unit 5 and a processing unit 6. On the measuring unit 5 according to the invention, the hollow spaces 16 correspond to the feed and discharge lines 39 of a standard device 33 and preferably all run out in a common connection plane 38′. Preferably, all these media connections are arranged in a straight line (cf. e.g. EP 1 260 265 B1). The following connections or fluid sources are each illustrated here by way of example (in order, from top to bottom):
A=agitation pressure for moving the liquids relative to samples immobilised on the object carriers 35 and chamber pressure for preventing gas bubbles; with an inlet valve and open outlet;
B=flow of drying gas (preferably N₂) with inlet valve;
C=liquid feed flow with inlet valve;
D=liquid discharge flow with outlet valve.

In principle, the processing units 6 of these three measuring designs are alike and comprise a serial/parallel data bus 8; a direct digital input/output line 9, an analogue/digital (A/D) converter 10 and a microcontroller 11 which communicate with one another or exchange data or signals via connecting lines 7.

FIGS. 4A, 4B and 4C each show a first pressure sensor 20 which is accomplished for measuring relatively low pressures (e.g. 10 mbar to 1,500 mbar) which are provided by the fluid source A. The pressure measurement signals are forwarded to the microcontroller 11 via the A/D converter 10 for evaluation.

FIGS. 4A, 4B and 4C each show a second pressure sensor 20 which is accomplished for measuring relatively high pressures (e.g. 1.5 to 3.5 bar) which are provided by the fluid source B. The pressure measurement signals are again forwarded to the microcontroller 11 via the A/D converter 10 for evaluation. The three measuring designs differ here in that this second pressure sensor 20 can be separated in FIGS. 4B and 4C from the source B by a valve; this is not the case in FIG. 4A.

FIG. 4C shows a first flow sensor 19 which is accomplished exclusively for measuring the flow of gas which is provided by the fluid source B. The gas flow measurement signals are forwarded to the microcontroller 11 via the serial/parallel data bus 8 for evaluation.

FIG. 4C shows a second and third flow sensor 19 which are in direct fluid connection with each other and which are accomplished exclusively for measuring the flow of liquid which is provided by the fluid source C and which leaves the measuring unit 5 via the liquid discharge flow D. The liquid flow measurement signals are also forwarded to the microcontroller 11 via the serial/parallel data bus 8 for evaluation. In contrast thereto, FIGS. 4A and 4B show a second and third flow sensor 19 which are also in direct fluid connection with each other, but are embodied for measuring a flow of liquid and a flow of gas, the flow of liquid being provided by the fluid source C, while the flow of gas is provided by the source B. In any case, these media leave the measuring unit 5 via the liquid discharge flow D.

All three variants according to FIGS. 4A, 4B and 4C comprise at least one temperature sensor 24 which is arranged independently of all these flows of fluid and connected to the direct digital input/output line 9 and the microcontroller 11 of the computer 12 via separate connecting lines 7.

Further embodiments, in which the measuring unit 5 of the measuring apparatus 4 is integrated into a hybridisation system 2 from the prior art, will be presented and commented on in greater detail hereinafter. As stated above and in the cited documents, a hybridisation system 2 of this type comprises at least one hybridisation unit 3. This hybridisation unit 3 provides the reaction room 34 which is defined by at least one standard device 33 and an object carrier 35. The principle is that the measuring apparatus 4 determines, when it is integrated into the hybridisation system 2 via its measuring unit 5, “on site” the actual values of the fluid parameters that are provided by the hybridisation system. In a preferred embodiment, the measuring unit 5 comprises substantially the same connections for the feeding and discharging of fluids 13 provided by the hybridisation system 2 as the standard device 33. Furthermore, the measuring unit 5 is embodied in its basic dimensions in such a way that it can be inserted into a hybridisation unit 3 instead of the standard device 33. In the dimensions of the hollow spaces 16 too, a measuring unit 5 corresponds substantially to those dimensions of the hollow spaces of a standard device 33. In addition, the fluid parameters measured in the measuring unit can be mathematically corrected or adjusted in such a way that they may be regarded as having been measured as under “real-time conditions” and can be compared to the situations in a reaction room 34 of the standard device 33. The fluid parameters measured in the measuring unit 5 are thus transferable to the fluid parameters of a hybridisation reaction.

In a preferred variant of this embodiment, the measuring unit 5 of the measuring apparatus 4 is embodied in such a way that the measuring unit can be inserted into a hybridisation system 2 instead of a first standard device 33′. In a hybridisation system 2 of this type, the standard device 33 of the hybridisation unit is accomplished as this first standard device 33′. The first standard device 33′ defines in combination with an object carrier 35 a single hybridisation room 34. A first standard device 33′ of this type is illustrated in FIG. 5A at position I inserted into the holder 26 of the hybridisation system. The measuring unit 5 is embodied in such a way that it can be inserted into the hybridisation system 2 instead of a further first standard device 33′. A situation of this type is illustrated in FIG. 5A at position II in the holder 26 of the hybridisation system 2. At this position II, no hybridisation reactions are thus carried out, as the measuring unit 5 according to this variant does not comprise or define a reaction room 34. The further positions III and IV are also occupied by further first standard device 33′. Thus, up to three hybridisation reactions can also be carried out in parallel with the determination of fluid parameters. Preferably, no object carrier 35 at all or, if so, an object carrier without immobilised samples is applied at the position II. The hybridisation rooms 34 at the positions I, III and IV are indicated by the sealing faces 50.

In a further preferred variant of this embodiment, the measuring unit 5 of the measuring apparatus 4 can be inserted instead of a second standard device 33″ of a hybridisation system 2. In this case, the standard device 33 of a hybridisation unit 3 of the hybridisation system 2 is embodied as this second standard device 33″. The second standard device defines in combination with an object carrier 35 at least two hybridisation rooms 34. These two hybridisation rooms 34 are sealed from the environment by means of two sealing faces of the second standard device 33″. A second standard device 33″ of this type is described in document EP 1 614 466 A2 and illustrated in FIG. 5B at position I inserted into the holder 26 of the hybridisation system. According to document EP 1 614 466 A2, a second standard device 33″ of this type can comprises a common agitation device 42 for both hybridisation rooms 34 as well as common connections for the individual lines 39. However, alternatively, and preferably for the use of a measuring unit 5 according to the invention, this second standard device 33″ has for each hybridisation space individual connections which lie in a common connection plane 38. A measuring unit 5 of this variant is embodied in such a way that it can be inserted into the hybridisation system 2 instead of a second standard device 33″. This situation is shown in FIG. 5B at position II. In this case, the measuring unit 5 also comprises, in addition to the measuring block 15 without a reaction room 34 and the at least one sensor 17, a reaction block 40. This reaction block 40 defines with the object carrier 35 at least one hybridisation room 34 which is delimited from the environment, preferably by means of a sealing face 50. The reaction block 40 comprises substantially the same lines 39″ as the second standard device 33″ for a hybridisation room 34 as well as substantially the same connections 38″ for the feeding and discharging of fluids 13 provided by the hybridisation system 2 as the second standard device 33″.

In this way, it is possible to guide in a measuring unit in parallel:
a) fluids 13 of the hybridisation system 2 into a reaction room 34 of the reaction block 40 of the measuring unit 5 for carrying out a hybridisation reaction; and at the same time
b) fluids 13 of the hybridisation system 2 into a measuring block 15 of the measuring unit 5 for determining fluid parameters provided by the hybridisation system 2.

If larger numbers of hybridisation reactions are to be carried out in addition and contemporaneously to a parameter measurement, in a hybridisation system 2, this embodiment of a measuring apparatus is used preferably in a hybridisation system 2 comprising at least one group made up of four hybridisation units 3, each with a second standard device 33″. The measuring unit 5 is then inserted into the hybridisation unit 3 instead of a second standard device 33″. FIG. 5B illustrates such a group of four hybridisation units which are inserted into a holder 26 of the hybridisation system 2. The second standard device 33″ of the hybridisation unit 3 at position II is replaced by a measuring unit 5. If a reaction is to be carried out by means of the reaction block 40 of the measuring unit 5 at this position II, an object carrier 35 with immobilised samples has to be positioned here accordingly.

The arrangement of four hybridisation units to form a group according to FIGS. 5A and 5B is preferred insofar as the temperature control plate of the hybridisation system 2 has dimensions such that a frame 28 the size of a microplate with four object carriers 35 arranged in parallel to one another just fits onto the temperature control plate. All the hybridisation rooms 34 of such a hybridisation unit 3 thus have identical temperature conditions. Depending on requirements, not just one of the first or second standard devices 33′,33″ is replaced by a measuring unit 5 according to the invention, but several of them are. If, for example, the hybridisation system 2 is to be adjusted for the first time after production, all the standard devices 33′,33″ are preferably replaced by measuring units 5. In this way, the fluid parameters can be determined and if appropriate adjusted at each of the positions I-IV.

In a further, preferred variant of this embodiment, the measuring unit 5 is embodied in such a way that it is inserted into the hybridisation unit 3 of a hybridisation system 2 instead of a third standard device 33′″. In a hybridisation system 2 of this type, the standard device 33 is accomplished as a third standard device 33′″. The third standard device 33′″ defines in combination with an object carrier 35 at least three or more hybridisation rooms 34. A third standard device 33′″ of this type is illustrated in FIGS. 5C and 5D, in each case at the position I/II. The individual reaction spaces are in this case delimited from the environment by separate sealing faces 50. The third standard devices 33′″ shown here each comprise four hybridisation rooms 34. As indicated in FIGS. 5C and 5D, a third standard device 33′″ is embodied so as to be larger in its basic dimensions than a first or second standard device 33″ (cf. with FIGS. 5A and 5B). More precisely, a third standard device 33′″ corresponds in its dimensions substantially to two interconnected first or second standard devices 33′,33″. This increase in size is preferred in order to make allowance for the increased number of necessary connections as well as feed and discharge lines of fluids 13 provided by the hybridisation system 2 and the feed lines thereof to or the discharge lines thereof from the four hybridisation rooms 34. The third standard device 33′″ can comprise common connections for the feeding and discharging of media or, for each hybridisation room 34 defined by the standard device, a separate set of connections for the feeding and discharging of fluids 13.

In this further variant, the measuring unit 5 is embodied in such a way that it can be inserted into the hybridisation system instead of the third standard device 33′″. A situation of this type is illustrated in FIGS. 5C and 5D, in each case at position III/IV in the holder 26 of the hybridisation system 2. In this case, this measuring unit 5 comprises a measuring block 15 without a reaction room 34 and with at least one sensor 17 as well as a reaction block 40. The reaction block 40 defines with the object carrier 35 at least two or more hybridisation rooms 34. The number of hybridisation rooms 34 defined by the reaction block 40 is variable. FIG. 5C illustrates at position III/IV a measuring unit 5, the reaction space of which defines three hybridisation rooms 34. FIG. 5D, on the other hand, shows at position III/IV a measuring unit 5, the reaction block of which defines four hybridisation spaces. In this case, all the hybridisation rooms 34 of a reaction block 40 are defined preferably with a single object carrier 35.

If larger numbers of hybridisation reactions are to be carried out in addition and contemporaneously to a parameter measurement in a hybridisation system with a third standard device 33′″, the measuring apparatus 4 is used preferably in a hybridisation system 2 comprising at least one group made up of two hybridisation units 3, each with a third standard device 33′″. The measuring unit 5 of the measuring apparatus 4 is then inserted into the hybridisation unit instead of a third standard device 33′″. FIGS. 5C and 5D show a group of this type made up of two hybridisation units 3 which are inserted into a holder 26 of the hybridisation system 2.

In a variant of these preferred embodiments, the reaction block 40 of the measuring unit 5 comprises, for replacing the second or third standard device 33″,33′″, further elements of these standard devices 33 for carrying out hybridisation reactions. Preferably, the reaction block 40 comprises in this case at least one specimen supply line 41 for the feeding of specimen liquids into at least one hybridisation room 34. Particularly preferably, the reaction block 40 furthermore comprises at least one agitation device 42,42′ for generating an agitation pressure and for moving liquids 13 in a reaction room 34. This agitation device 42 of the reaction block 40 is constructed substantially like that of the standard device 33. Depending on the standard device 33 which is replaced by the reaction block 40 of a measuring unit 5, the reaction block can also comprise a number of agitation devices 42 that corresponds to the number of hybridisation rooms 34.

Particularly preferably, the reaction block 40 comprises at least one pressure means 47, which is completely separate from the agitation device 42, for building up a room pressure to be superimposed by the agitation pressure in at least one hybridisation room 34. The specimen supply line 41, agitation device 42 and pressure means 47 have already been discussed in relation to FIG. 1 and are known from documents EP 1 260 265 B1 or EP 1 614 466 A2. These means will therefore not be commented on again at this point. The important thing is that the principle of the standard device is transferable to a reaction block 40 of the measuring apparatus 4 according to the invention. On account of the resulting complexity and the ensuing lack of clarity of the drawings showing a measuring unit 5 with a measuring block 15, its hollow spaces 16 and sensors 17 and additionally with the reaction block 40, specimen supply line 41, agitation device 42 and pressure means 47, reference will be made here merely by way of example to a basic arrangement and a drawing of this type will be dispensed with. Preference is given to a measuring apparatus 4 with at least one measuring unit 5 comprising at least one sensor for determining a chamber pressure. This chamber pressure serves to prevent the formation of gas bubbles in the reaction room 34 and is generated by the pressure means 47 (cf. source A in FIG. 4). A sensor used by way of example is the above-described low-pressure sensor which is used to determine the chamber pressure and also an agitation pressure superimposed, preferably cyclically, thereon.

Particularly preferred variants of the described embodiments comprise a temperature control plate which is embodied as a bottom plate. A temperature plate of this type is thus capable of receiving up to four object carriers 35 in a surface-contacting manner. This may be desirable to prevent a loss of temperature between the temperature control plate and object carrier 35. In a further possible variant, the temperature control plate is embodied as a cover plate (not shown) which can be used to lower up to four object carriers 35 onto standard devices 33 or measuring units 5 which are turned round (compared to FIGS. 1 and 2). It is also possible for up to four object carriers 35 to be raised onto standard devices 33 or measuring units 5 which are oriented normally (compared to FIGS. 1 and 2) (not shown).

It should be emphasised that a measuring apparatus 4 according to the invention can be used to determine both snapshots of the parameters prevailing in a laboratory system 1 and also the behaviour of the parameters as a function of time. In the latter measurement, sensor data are continuously monitored and if appropriate recorded over a specific period of time. It is then possible to read off, for example, from these data the pressure curve or the march of temperature for a desired period of time. It is also conceivable, on use of two or more sensors for a specific fluid parameter, gradients may be detectable within the system. The determination of pressure gradients or else temperature gradients is in this case advantageous.

The present invention includes, in addition to a measuring apparatus 4, also a method for determining fluid parameters provided by a laboratory system 1. The carrying-out of such a method according to the invention comprises using a measuring apparatus 4 with a measuring unit 5 and a processing unit 6 which have already been discussed hereinbefore in detail. Particularly preferably, the measuring apparatus 4 is incorporated into a hybridisation system 2. The method according to the invention as well as the measuring apparatus 4 according to the invention can be used at various levels of use of a laboratory system 1. For example, it can be introduced immediately during production for the setting and checking of the newly finished laboratory system 1 to defined and standardised factory settings. For this application, it is preferable that sensors 17 to be used are checked in view of their functioning by means of calibration sensors (e.g. the sensors F-20/CV-5k0-ABD-33-V and L23-ABD-33-K-70S from the company Bronkhorst, Nenzlingerweg 5, 4153 Reinach, Switzerland), firstly externally. If the laboratory system 1 is operative on a customer's premises, both customers and service engineers can themselves monitor, by means of the measuring apparatus 4, the fluid parameters which are actually provided by the laboratory system. Thus, it is now possible to bypass long-winded test experiments, which are usually based on biological or chemical assays, thus allowing a saving of up to one third of the test time which had previously to be used. A large part of the test costs can thus be saved. In addition, a check of the preset parameters can be carried out, before e.g. particularly cost-intensive experiments are to be carried out at the laboratory system 1. E.g. an object carrier costs about CHF 1,000, so that, on use of 4 to 40 object carriers for each test series, allowance must be made for up to CHF 40,000. Misdiagnoses caused by apparatus deficiencies may also be reduced, for example in diagnostic laboratories (e.g. of clinics), by regularly checking the settings of the systems by means of a measuring apparatus 4 according to the invention. In this way, it is possible to identify and to correct, in the event of dubious or critical reaction results, in a simple method malfunctions or miscalibrations of the laboratory system 1 used. In the event of dubious or critical reaction results, the possibility of restricting the search for errors by means of the measuring apparatus 4 according to the invention at an early stage in the error analysis is to be regarded as being particularly advantageous. It is thus possible, on use of the measuring apparatus 4, to reliably allocate as early as in a first step an error to the apparatus (if there is an apparatus deficiency) or, if no apparatus error may be ascertained, to the application.

Preferably, this method is used for calibrating and/or adjusting the laboratory system 1 by means of the signals which are received by the at least one sensor 17 of the measuring unit 5 and processed by the processing unit 6. In this case, the term “calibrating” is intended to refer in this connection to the collecting of measured data and the comparing of these data to defined standards. The term “adjusting” refers in this connection accordingly to the collecting of data, the comparing with the standard and the adjusting.

Unless otherwise stated, the features and embodiments of the invention that are presented here can be combined with one another to form all manner of variants. The resulting embodiments come under the scope of the present invention.

### LIST OF REFERENCE NUMERALS

1 laboratory system
2 hybridisation system
3 hybridisation unit
4 measuring apparatus
5 measuring unit
6 processing unit
7 connection between 17 and 6
8 serial/parallel data bus
9 direct digital input/output line
10 A/D converter
11 microcontroller
12 computer
13 fluids
15 measuring block
16,16′hollow spaces
17 sensor
19 flow sensor for liquid/gas
20 pressure sensor for liquid/gas
21 light barrier
22 printed circuit board
23 metal plate
24 temperature sensor
25 niche
26 holder
28 frame
29 axis 46′ agitation line of 42
30 connecting plate
31 surface of 2
32 spring/spring element
33 standard device
33′ first standard device
33″ second standard device
33′″ third standard device
34 hybridisation room/reaction room
35 object carrier
36 surface of the object carrier
37 connection plate of 3
38 common connection plane of the feed/discharge lines of 33
38′ common connection plane of the hollow spaces of 5
38″ common connection plane of the feed/discharge lines of 40
39 feed/discharge lines of 33
39′ feed/discharge lines of 2
39″ feed/discharge lines of 40
40 reaction block
41 specimen supply line
42 first agitation device
42′ second agitation device
43 membrane of 42
43′ membrane of 42′
44 pressure chamber of 42
44′ pressure chamber of 42′
45 agitation chamber of 42
45′ agitation chamber of 42′
46 agitation line of 42
46′ agitation line of 42
47 pressure means
49 heating element
50 sealing face
51 base plate of 3
52 surface of 51

What is claimed is:

1. Measuring apparatus (4) with a measuring unit (5) and a processing unit (6) for determining fluid parameters provided by a laboratory system (1), the measuring unit (5) being accomplished to be integratable into this laboratory system (1),
wherein
(a) the measuring unit (5) comprises a measuring block (15) without a reaction room (34) and at least one sensor (17);
(b) the measuring block (15) comprises hollow spaces (16) for receiving or guiding fluids (13) provided by the laboratory system (1), the hollow spaces (16) being arranged substantially completely within the measuring block (15); and
(c) the at least one sensor (17), for determining physical and/or chemical parameters of fluids (13) located in the hollow spaces (16), is arranged on or in fluidic operative connection with these hollow spaces (16) of the measuring block (15).

2. Measuring apparatus (4) according to claim 1, wherein the at least one sensor (17) is accomplished for determining a flow rate of a fluid or for determining a fluid pressure.

3. Measuring apparatus (4) according to claim 1, wherein the measuring unit (5) comprises at least two sensors (17), of which the first sensor (19) is accomplished for determining a flow rate of a fluid and the second sensor (20) is accomplished for determining a fluid pressure.

4. Measuring apparatus (4) according to claim 1, wherein the measuring unit (5) comprises, for determining a flow rate of a fluid, at least two identically constructed sensors (17) which are arranged set apart from one another on or in fluidic operative connection with a fluid line.

5. Measuring apparatus (4) according to claim 4, wherein the at least two identically constructed sensors (17) are accomplished as flow sensors (19) or as light barriers (21).

6. Measuring apparatus (4) according to claim 1, wherein the measuring unit (5) of the measuring apparatus (4) is accomplished to be insertable into a hybridisation system (2).

7. Measuring apparatus (4) according to claim 1, wherein the measuring unit (5) of the measuring apparatus (4) is accomplished to be insertable into a hybridisation system (2), and wherein the measuring unit (5) of the measuring apparatus (4) comprises a niche (25) in which at least one temperature sensor (24) is arranged, the measuring unit (5) and a surface (31) of the hybridisation system (2), from which a temperature is to be determined, being accomplished so as to be movable relative to each other.

8. Measuring apparatus (4) according to claim 7, wherein the at least one temperature sensor (24) of the measuring unit (5) is accomplished to be able to be abutted resiliently to a surface (31) of the hybridisation system (2), from which a temperature is to be determined.

9. Hybridisation system (2) with at least one hybridisation unit (3), this hybridisation unit (3) comprising a standard device (33) which defines in combination with an object carrier (35) at least one hybridisation room (34), this standard device (33) comprising connections for the feeding and discharging of fluids (13) provided by the hybridisation system (2), wherein the hybridisation system (2) comprises at least one measuring unit (5) according to claim 1, the measuring unit (5) comprising substantially the same connections for the feeding and discharging of fluids (13) provided by the hybridisation system (2) as the standard device (33) and being accomplished in its basic dimensions in such a way that the measuring unit (5) is accomplished to be insertable into a hybridisation unit (3) instead of the standard device (33).

10. Hybridisation system (2) according to claim 9, the standard device (33) being accomplished as a first standard device (33′), wherein the measuring unit (5) is accomplished to be insertable instead of the first standard device (33′).

11. Hybridisation system (2) according to claim 9, the standard device (33) being accomplished as a second standard device (33″) which defines in combination with an object carrier (35) at least two hybridisation rooms (34), wherein the measuring unit (5) comprises a measuring block (15) without a reaction room (34) and with at least one sensor (17) as well as a reaction block (40), the reaction block (40) defining with the object carrier (35) at least one hybridisation room (34), and the measuring unit (5) being accomplished to be insertable instead of the second standard device (33″).

12. Hybridisation system (2) according to claim 9, the standard device being accomplished as a third standard device (33′″) which defines in combination with an object carrier (35) at least three or more hybridisation rooms (34), wherein the measuring unit (5) comprises a measuring block (15) without a reaction room (34) and with at least one sensor (17) as well as a reaction block (40), the reaction block (40) defining with the object carrier (35) at least two or more hybridisation rooms (34), and the measuring unit (5) being accomplished to be insertable instead of the third standard device (33′″).

13. Hybridisation system (2) according to claim 10, wherein it comprises at least one group made up of four hybridisation units (3), at least one measuring unit (5) being inserted into a group of hybridisation units (3) instead of a first or second standard device (33′,33″).

14. Hybridisation system (2) according to claim 11, wherein the reaction block (40) of the measuring unit (5) comprises, for replacing the second or third standard device (33″,33′″), at least one specimen supply line (41) for the feeding of specimen liquids into at least one hybridisation room (34).

15. Hybridisation system (2) according to claim 11, wherein the reaction block (40) of the measuring unit (5) comprises, for replacing the second or third standard device (33″,33′″), at least one agitation device (42) for moving liquids in at least one hybridisation room (34), the agitation device (42) comprising at least one membrane (42) which separates a pressure chamber (44), in which an agitation pressure can be generated, from an agitation chamber (45) which is connected to at least one hybridisation room (34) via an agitation line (46).

16. Hybridisation system (2) according to claim 15, wherein the reaction block (40) of the measuring unit (5) comprises, for replacing the second or third standard device (33″,33′″), at least one pressure means (47), which is completely separate from the agitation device (42), for building up a room pressure in at least one hybridisation room (34), this room pressure (42) being superimposed by the agitation pressure.

17. Hybridisation system (2) according to claim 9, wherein the measuring unit (5), which comprises at least one temperature sensor (24), and a surface (31) of the hybridisation system (2) are accomplished so as to be movable relative to each other, the surface (31) being accomplished as a temperature control plate.

18. Hybridisation system (2) according to claim 17, wherein the temperature control plate is accomplished as a bottom plate or as a cover plate.

19. Hybridisation system (2) according to one of claims 15, wherein at least one sensor (17) is embodied for determining a chamber pressure.

20. Method for determining fluid parameters provided by a laboratory system (1) using a measuring apparatus (4) according to claim 1 with a measuring unit (5) and a processing unit (6), the measuring unit (5) being integrated into this laboratory system (1), wherein
(a) the measuring unit (5) comprises a measuring block (15) without a reaction room (34) and at least one sensor (17);
(b) the measuring block (15) comprises hollow spaces (16) which receive fluids (13) provided by the laboratory system (1) and which are arranged substantially completely within the measuring block (15); and
(c) physical and/or chemical parameters of the fluids (13) located in the hollow spaces (16) are determined by the at least one sensor (17) which is arranged on or in fluidic operative connection with these hollow spaces (16) of the measuring block (15).

21. Method according to claim 20, wherein a pressure and/or a flow rate of a fluid (13) located in the hollow spaces (16) of the measuring block (15) is measured using the at least one sensor (17).

22. Method according to claim 20, wherein a flow rate of a fluid (13) is measured using at least two identically constructed sensors (17), the at least two identically constructed sensors (17) being arranged set apart from one another on or in fluidic operative connection with a fluid line.

23. Method according to claim 20, wherein signals received by the at least one sensor (17) of the measuring unit (5) and processed by the processing unit (6) are used for calibrating and/or adjusting the laboratory system (1).

24. Method according to claim 20, wherein it is carried out for determining physical and/or chemical fluid parameters provided by a hybridisation system (2).

25. Method according to claim 24, wherein a temperature of a surface (31) of the hybridisation system (2) is determined using a temperature sensor (24) arranged in a niche (25) of the measuring unit (5), the measuring unit (5) and the surface (31) of the hybridisation system (2) being movable relative to each other.

26. Method for determining fluid parameters provided by a hybridisation system (2) using a measuring apparatus (4) according to claim 1, wherein at least one hybridisation unit (3) of the hybridisation system (2) comprises a standard device (33) which defines in combination with an object carrier (35) at least one hybridisation room (34), wherein fluids (13), which are provided by the hybridisation system (2), being fed into and/or discharged from the standard device via connections thereof, wherein a measuring unit (5) of the measuring apparatus (4) is inserted into a hybridisation unit (3) instead of the standard device (33), the measuring unit (5) comprising a measuring block (15) without a reaction room (34) and at least one sensor (17), and the measuring unit (5) comprising substantially the same connections for the feeding and discharging of fluids (13) provided by the hybridisation system (2) as the standard device (33) and being accomplished in its basic dimensions like this standard device (33).

27. Method according to claim 26, wherein at least one measuring unit (5) is inserted instead of a first standard device (33′), the first standard device (33′) defining at least one hybridisation room (34) in combination with an object carrier (35).

28. Method according to claim 26, wherein at least one measuring unit (5) is inserted instead of a second standard device (33″), the measuring unit (5) furthermore comprising a reaction block (40), the reaction block (40) defining with the object carrier (35) at least one hybridisation room (34) and the at least one sensor (17) determining the physical and/or chemical fluid parameters at a separate location from but simultaneously with a hybridisation reaction.
